(19) United States
(12) Patent Application Publication
(10) Pub. No.: US 2019/0099610 A1
(43) Pub. Date: Apr. 4, 2019

(54) CANCER CELL TREATMENT

(71) Applicant: Hyper Light Technologies, LLC, Nashville, NC (US)

(72) Inventor: Kurt A. Garrett, Raleigh, NC (US)

(73) Assignee: Hyper Light Technologies, LLC, Nashville, NC (US)

(21) Appl. No.: 15/724,575

(22) Filed: Oct. 4, 2017

(51) Int. Cl. A61N 5/06; A61B 18/22

(52) U.S. Cl. CPC A61N 5/0601; A61N 5/0616; A61B 2018/2222; A61N 2005/0661; A61B 18/22

(57) ABSTRACT

A method of killing cancer cells quickly without damaging cell structures and doing little or no damage to surrounding tissue. A high energy UV light with little to no heat is utilized.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a photodynamic method and devices for the treatment of cancer. In particular, it relates to inhibition and death of cancer cells using a photodynamic device.

### Description of Related Art

The treatment of cancer is still a major medical concern since it is a disease which has many forms and has a high chance of afflicting the majority of the population at some point in their lives, for which many people still die or suffer permanent damage.

The treatment of cancer has taken many forms including surgery and chemotherapy, both of which have serious drawbacks and limitations as to use of this modality. Photodynamic therapy, which involves light in some form, is a more recent development. This is a method of directly or indirectly treating cancer cells using either direct treatment of the cells by a light or the administration of a compound which is activated by light. Accumulation of photosensitive materials has its difficulties since these compounds also accumulate in healthy tissue. Use of LED blue light directly has been utilized but because of low power it is limited to treating top layer cells, such as blood or skin type cancers. Higher intensity lights are available but due to heat issues the effectiveness of these types of lights is unknown since heat will destroy healthy tissue. Accordingly, it is unclear if light therapy can advance past current technologies. However, more effective therapies would be welcome in the treatment of cancer.

### BRIEF SUMMARY OF THE INVENTION

The present invention relates to the discovery that high intensity lights which have a substantial portion of the heat removed, such as described in US Patent publication number US2017/0028089 published on Feb. 2, 2017 in the name of Kurt A. Garrett and U.S. patent application Ser. No. 15/712,559 filed on Sep. 22, 2017 in the name of Kurt A. Garrett, incorporated herein by reference, can kill or inhibit cancer cells without damaging the underlying cell structures of the cancer cells or those of nearby heathy cells, thus allowing any damaged healthy cells to recover. While these devices are shown in the incorporated references to kill microorganisms, it has been surprisingly found that the same light can kill a cancer cell without destroying the cell supporting structures and thus do less damage to surrounding healthy cells and in most cases allow damaged healthy cells to regenerate. Further, it is discovered that the method can be utilized not only on the surface of cells but can be used to penetrate deep into a tumor with the aid of light delivery devices, such as light fibers or other thin light tube type devices.

In one embodiment, there is a method of inhibiting or killing cancer cells comprising:
a) selecting a high intensity UV light source that delivers a low heat light to the end of a light guide; and
b) exposing the cancer cells to the light from the end of the light guide at sufficient distance and time to kill or inhibit the cancer cell.

In another embodiment, there is a device for inhibiting or killing cancer cells on the inside of a cancerous tumor using a light device having a high intensity UV light source that delivers a low heat light to the end of a light guide, the light device having a fiber light guide positioned at the end of the light guide that can be inserted into the cancerous tumor to deliver the low heat light to the inside of the cancerous tumor.

### BRIEF DESCRIPTION OF THE DRAWINGS

FIG. 1 depicts the use of a device in killing cancer tumor on a substrate.

FIG. 2 is a view of an optic fiber inserted into a tumor and optionally into the animal to reach the inside of a tumor.

FIGS. 3a and 3b are views of an optical fiber attached to the end of a light tube.

FIGS. 4a and 4b are photos of control, treated, and untreated mouse at 11 and 16 days showing only tumor growth in the untreated mouse.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “inhibiting or killing cancer cells” refers to a living and multiplying cancer cell either being alive and unable to replicate, or outright killed. However, underlying structures are left essentially intact and surrounding non-cancerous tissue spared and either not injured at all or capable of regeneration and/or repair.

As used herein, the term “high intensity UV light source that delivers a low heat UV light” refers to a device that has a light source producing a spectrum of UV light capable of killing a microorganism, such as taught in US Patent publication number US2017/0028089 published on Feb. 2, 2017 in the name of Kurt A. Garrett and U.S. patent application Ser. No. 15/712,559 filed on Sep. 22, 2017 in the name of Kurt A. Garrett. In particular, it produces a wide UV spectrum (i.e. more than just an isolated wavelength) even though it can produce other spectrums of light and, in one embodiment, the light produces a high UV output. It consists of a high intensity light and a dichroic reflector which focuses the light and removes heat and then delivers the UV light to a light tube where the light coming out of the far end of the light tube is used to kill cancer cells without damaging underlying physical structures thus minimizing or eliminating damage to healthy cells near the cancer cells.

As used herein, the term “high intensity light” refers to a bulb of any kind which produces a sterilizing UV light. This can be UVA, UVB, UVC, or a combination. Regular bulbs, but also high intensity discharge (HID) bulbs, are also embodiments of the invention. For example, a high intensity mercury xenon (HgXe) bulb can be utilized. These types of bulbs are high UV output bulbs. In general, the light output of some bulbs of the invention is from about 0.1 J/cm² to about 50.0 J/cm². It also includes arc type lamps when they are focused properly to the light tube.

As used herein, the term “high intensity light” refers to light output of about at least 80 lumens per watt output. In order to achieve this high intensity light output, one cannot use low or medium pressure lamps that produce UV light, as they do not produce enough light output. In order to achieve the high intensity output needed, one can add to the arc discharge lamp's light output an elliptical reflector which collimates the polychromatic light into still greater intensity (intensity being understood as energy per area) of about 100 lumens per watt (i.e. producing the high intensity light output needed).

As used herein, the term “dichroic reflector” refers to any of a dichroic focus, reflector, mirror, lens, or the like that takes light from the light source and allows some or all of the thermal energy to pass through the reflector while taking the light, especially the UV light, to be reflected for focusing. In one embodiment, there may be more than one dichroic reflector but at least one must focus the light to the light pipe. The dichroic reflector can be any shape that works to either remove heat or focus the light but, in one embodiment, it is an elliptical shape for focusing. In one embodiment, an elliptical dichroic reflector is used with an arc lamp. This is different from a dichroic filter which only filters or reflects light but does not pass heat wavelengths through it. The dichroic filter can be a powered or unpowered device.

As used herein, the term “polychromatic” refers to light comprising multiple wavelengths of light.

As used herein, the term “sufficient distance and time” refers to the time period and distance from the tumor that light produced by the device is exposed (light shining on it) to a cancer cell in order to kill it. In one embodiment, it is from about 0.01 seconds to about 5 seconds. In one embodiment, a shutter is utilized to open, close, and modulate the passage of light from the light source to the cancer cell. The exposure can be directly from the end of the light tube or extended via a light fiber at the end of the light tube, especially for insertion of the fiber into a cancer cell tumor either directly or through the skin of the animal or human.

As used herein, the term “cancer cell, tumor, or tumor cells” refers to a cell undergoing early, intermediate, or advanced stages of multi-step neoplastic progression. This includes cells in early, intermediate, and advanced stages of neoplastic progression including “pre-neoplastic” cells (i.e., hyperplastic cells and dysplastic cells) and neoplastic cells in advanced stages of neoplastic progression of a dysplastic cell. Cancer cell also refers to a cancer cell that is translocated from a primary cancer site a location where the cancer cell initially formed from a normal, hyperplastic, or dysplastic cell) to a site other than the primary site where the translocated cancer cell lodges and proliferates.

As used herein, the term “tumor” refers to a plurality of cancer cells that may or may not be metastatic, such as ovarian cancer, breast cancer, lung cancer, prostate cancer, cervical cancer, pancreatic cancer, colon cancer, stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testicular cancer, kidney cancer, endometrial cancer, uterine cancer, bladder cancer, bone marrow cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), alioblastoma, lymphoma, and leukemia.

As used herein, the term “fiber light guide” refers to any fiber capable of carrying UV light of any kind from one end to the other and in one embodiment, carries light of at least 180 nm to 465 nm. Well known light fibers include those made of fused silica, pure silica, organosilicons, hollow tubes, clad and unclad fibers where the fibers are either singular or bundled. Other optical fibers include liquid fibers that are water based or other diluents such as alcohols, ethers, aldehydes, ketones, and other liquids suitable for transmitting effective wavelengths and some may reduce thermal energy including infrared energy.

In one embodiment, there is an interface device which allows for a plurality of different diameter fibers to attach to the end of the light tube. In one embodiment, the interface device is shown in the figures and how it would connect different fibers. Also shown is the connection to the end of the light tube.

Thus, it is one aspect of the present invention to provide an apparatus and method that will kill cancer cells and preserve underlying structures and do minimal damage to surrounding tissue by way of high-intensity polychromatic or broad spectrum light irradiation, including UV-irradiation. It is another aspect of the present invention to provide an apparatus using light that has been put into a light fiber for insertion into a human or animal, or into a tumor, to directly expose cells inside a tumor to the light of the invention. The cancer cells can be removed from the patient, from the surface of the patient or inside the patient, and as long as the light can reach those structures, as taught herein, the cancer cells can be killed without surrounding tissue damage of any significance.

One skilled in the art will appreciate that, for this and other processes and methods and devices disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Having described the present invention, various aspects of the invention having more specific embodiments and examples will now be described in greater detail by way of the following specific examples. These examples demonstrate quantitatively the effectiveness of the invention for killing cancer cells. In these examples, standardized cancer cells were specifically grown to be treated. The deliberate introduction of high counts of cancer cells to a product results in a high degree of self-shielding of the cancer cells/tumor. Therefore, these examples represent a dramatic demonstration of the effectiveness of the high-intensity light treatment of cancer.

In the method of the present invention, the device of the invention is used to expose cancer cells to light for sufficient distance and time to inhibit or kill the cancer cell(s). In one embodiment, a light fiber is inserted into a group of cells to get more direct exposure of a tumor to the effects of the light.

The results of testing indicate that cells are killed by DNA disruption but underlying structures are left intact. The exposure in the present invention is an amount determined to be relatively safe for surrounding non-cancerous cells.

### DRAWINGS

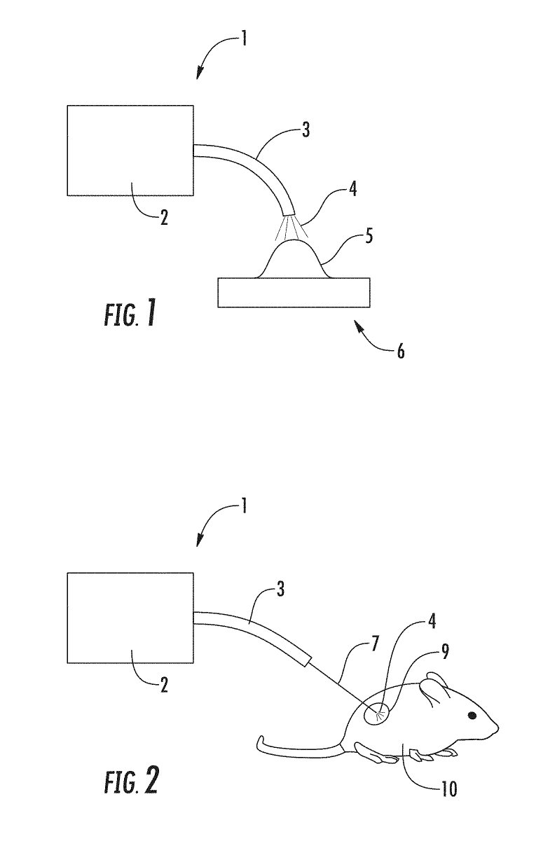

Now referring to the drawings, FIG. 1 is a light device delivering high intensity UV containing light to the end of a light guide wherein there is a low or no heat factor of the light in exposing tissue to the UV containing light. Device 1 consists of light generating device 2 (as for example shown in US Patent publication number US2017/0028089 published on Feb. 2, 2017 in the name of Kurt A. Garrett and U.S. patent application Ser. No. 15/712,559 filed on Sep. 22, 2017 in the name of Kurt A. Garrett) and a light guide 3 delivering a low heat UV light 4 to a tumor 5 on a substrate 6. The tumor cells are killed while the tumor structure is left intact other than cell death.

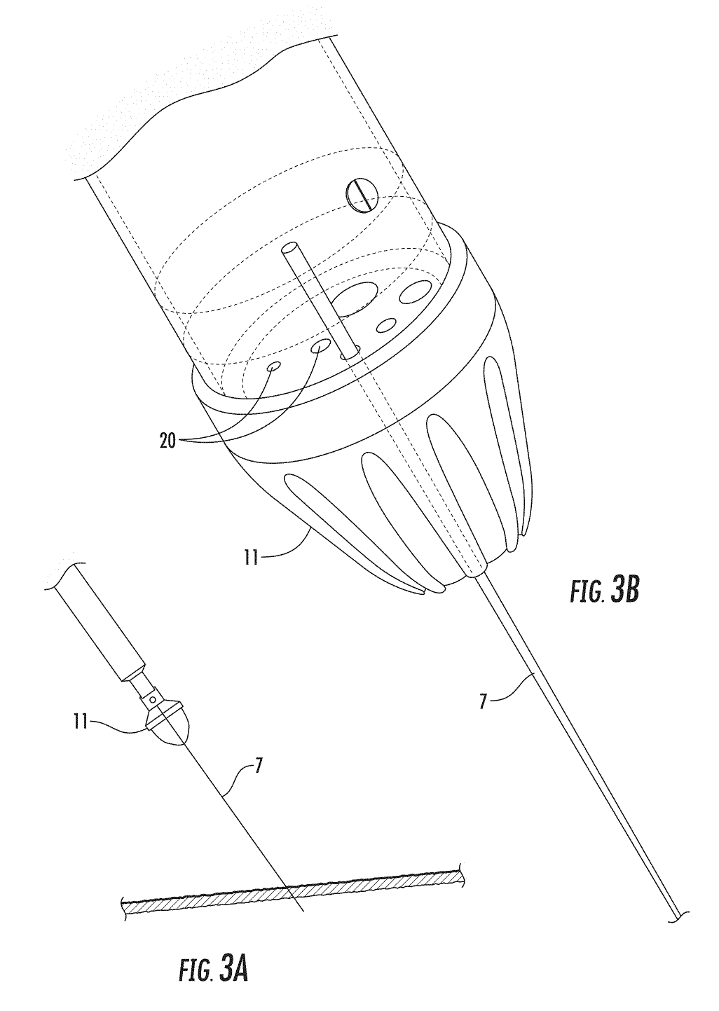

FIG. 2 shows a tumor 9 in a mouse 10. The device 1 has a light fiber 7 attached to the end of the light guide 3 and the light fiber 7 inserted into tumor 9 to deliver light 4 to the interior of the tumor.

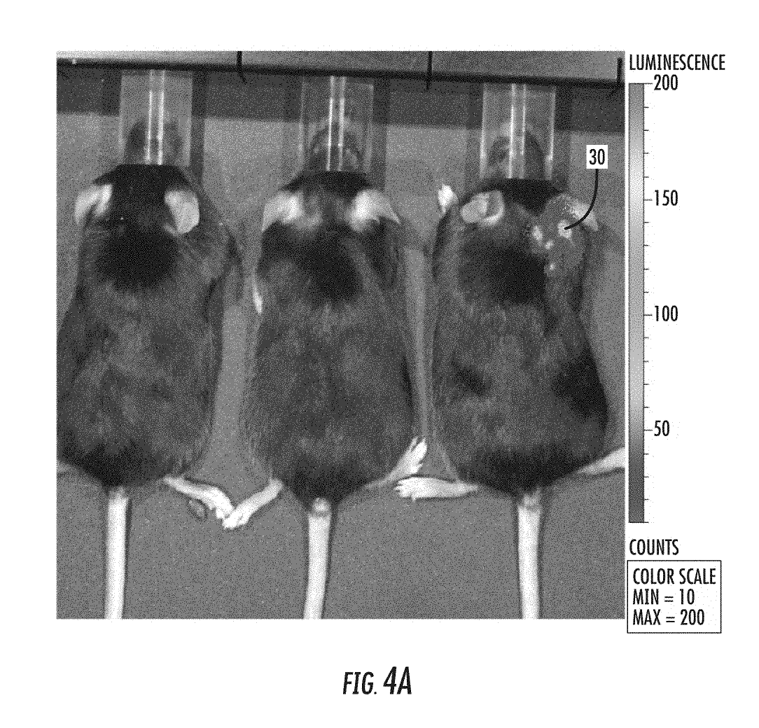

FIGS. 3a and 3b show a fiber connecting device 11 to attach a light fiber 7 to the end of the light guide. In FIG. 3b, it can be seen that the connector 11 can rotate and one can attach a number of different diameter fibers in fiber holes 20 for delivering differing amounts of light. One skilled in the art can design attachments and decide of fibers and diameters in view of this disclosure without undue experimentation.

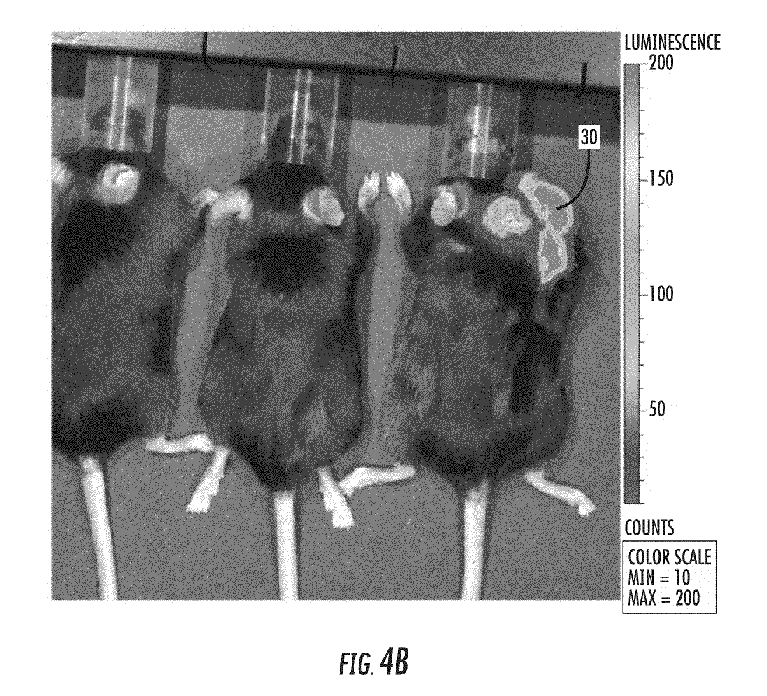

FIGS. 4a and 4b show from left to right an untreated rat, a rat given 380,000 metastatic melanoma cells and treated with UV high energy, low heat light, and a rat only treated with melanoma cells. On days 11 and 16 only the third rat developed a tumor 30.

### EXAMPLES

### Example 1

### Killing of Cancer Cells on a Substrate

Polystyrene well plates are commonly used to culture cells. The base of individual wells is made of polystyrene and hold microorganisms above the base of the floor of growth media. Cancer cells (350,000 metastatic melanoma cells) were transferred from a concentrated stock mixture and added to a well plate containing colored translucent liquid growth media. High intensity ultraviolet light, as described herein, was shone into the media containing the cancer cells (metastatic melanoma).

### Example 2

### Killing of Cancer Cells in an Animal Utilizing an Optical Fiber

Cancer cells can be killed in an animal by using the light energy described herein and directing it toward cancer cells using optical fibers. Various types of optical fibers can be employed to deliver high intensity light energy to cancer cells or tumors in animals, especially when not directly on the surface of a tumor. It is important to provide the beneficial light to the smallest number of plaques of cancer cells which have a tendency to multiply by exposing aggregated cancer cells (cancer tumor) to the same light using the facility of optical fibers which have the benefit of threading tumors and entering orifices of various sizes. Optical fibers are ideally suited for treating cancer cells that can range from invisible plaques to large tumors easily the size of a basketball. Killing tumors using the light described herein can be controlled by combining the following features into various configurations: 1) lamp wattage 2) light intensity (controlled by physical alignment) 3) exposure time and 4) type of optical fiber chosen.

### Example 3

### Killing of Cancer Cells in an Animal Utilizing Optical Lenses

Cancer cells may be killed by using optical lenses made of materials like quartz. Quartz transmits polychromatic light and can be shaped and polished to effectively focus polychromatic light onto cancer and thereby killing it in an animal. The efficiency of killing cancer using this method is affected by the penetration of the light on the cancer. Polychromatic light penetration containing UV light depends on the target material UV light absorption, the light distance from the target, the light intensity, the exposure time, and heat during the delivery or treatment. The facility of a wide range of optical fibers ranging from 10 microns through 600 microns can be used individually or bundled together to fill into the smallest spaces created naturally of artificially from blood capillaries to man-made endoscopes.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A method of inhibiting or killing cancer cells comprising:
a) selecting a high intensity UV light source that delivers a low heat UV containing light to the end of a light guide; and
b) exposing the cancer cells to the light from the end of the light guide at sufficient distance and time to kill or inhibit the cancer cell.

2. The method according to claim 1 wherein the cancer cells on the surface of a cancerous tumor are exposed to the light.

3. The method according to claim 1 wherein cancer cells on the inside of a cancerous tumor are exposed to the light by inserting a fiber light guide into the tumor.

4. A device for inhibiting or killing cancer cells on the inside of a cancerous tumor using a light device having a high intensity UV light source that delivers a low heat light to the end of a light guide the light device having a fiber light guide positioned at the end of the light guide that can be inserted into the cancerous tumor to deliver the low heat light to the inside of the cancerous tumor.

5. The device according to claim 4 wherein the fiber is selected from the group consisting of fused silica, quartz fiber, electrical conducting polymers fibers, carbon-polymer composite fiber, and liquid fiber.

6. A device according to claim 4 which further comprises a connecting device for interfacing with the device and connecting the fiber to the device the connecting device having an accommodation for a plurality of different diameter fibers.
